(19) United States
(12) Patent Application Publication
(10) Pub. No.: US 2026/0207323 A1
(43) Pub. Date: Jul. 23, 2026

(54) INTRAOCULAR LENS SUSPENSION STRUCTURE

(71) Applicants: The Eye Hospital of Wenzhou Medical University, Wenzhou (CN); The First People's Hospital of Aksu Prefecture, Aksu Prefecture (CN)

(72) Inventors: Zhong Lin, Wenzhou (CN); Ying Wang, Aksu Prefecture (CN); Chun Xiao, Aksu Prefecture (CN); Wei Lin, Wenzhou (CN)

(21) Appl. No.: 19/372,111

(22) Filed: Oct. 28, 2025

(30) Foreign Application Priority Data
Jan. 17, 2025 (CN) 202510077020.7

(51) Int. Cl. A61F 2/16

(52) U.S. Cl. CPC A61F 2/16; A61F 2002/1681; A61F 2220/0075; A61F 2250/0012

(57) ABSTRACT

An intraocular lens (IOL) suspension structure includes a first fixing plate and a second fixing plate, fixedly interconnected by guide columns. A first moving plate and a second moving plate, slidably matched with the guide columns, are arranged between the two fixing plates. Curved needles are arranged on a left end face of the first moving plate through clamping mechanisms, and the clamping mechanisms are arranged on a right end face of the second moving plate. Auxiliary holders are arranged between the first fixing plate and the second fixing plate. The IOL suspension structure is ingeniously designed, functionally practical, and structurally simple. It effectively solves the problem in existing devices where curved needles on a first movable stage often fail to realign with connecting tubes on a second movable stage after passing through the eyeball and an IOL, significantly compromising surgical efficiency.

### CROSS-REFERENCE TO RELATED APPLICATION

This application claims priority of Chinese Patent Application No. 202510077020.7, filed on January 17, 2025, the entire contents of which are incorporated herein by reference.

### TECHNICAL FIELD

The present disclosure relates to the field of medical devices, and in particular to an intraocular lens (IOL) suspension structure.

### BACKGROUND

IOL is a special lens made of synthetic materials, including silicone, polymethyl methacrylate (PMMA), and hydrogel. Functionally and structurally similar to the eye's natural lens, the IOL is characterized by its light weight, high optical performance, and absence of antigenicity, inflammogenicity, carcinogenicity, as well as being non-biodegradable. After the cloudy natural lens is removed during cataract surgery, the IOL is implanted into the eye to replace the original lens. This allows external light to focus properly on the retina, enabling the patient to see surrounding objects clearly.

A patent document with application number 202222149216.7 provides an IOL suspension structure. It includes two sets of curved needles for suspending the IOL and a telescoping mechanism. One end of each set of curved needles is connected by a suture. The telescoping mechanism includes a second fixing stage, a second movable stage, a first movable stage, and a first fixing stage arranged sequentially from left to right. The second movable stage is equipped with two sets of connecting tubes, each containing a plurality of sets of sealing plugs. The structure also includes a first sleeve rod connected to a second sleeve rod. An airbag is arranged on an inner wall of the first sleeve rod. Through the configuration of the telescoping mechanism and the curved needles, the two sets of curved needles enter the eyeball and pass through the IOL as a threaded rod rotates, achieving a four-point fixation. Furthermore, by arranging the second movable stage, the first and second sleeve rods, along with gears and racks, the retraction of the curved needles from the eyeball is achieved, while drawing the two sets of needles closer together to facilitate knotting and secure fixation.

However, during practical use, the curved needles on the first movable stage often fail to realign with the connecting tubes on the second movable stage after passing through the eyeball and the IOL, adversely affecting surgical efficiency. Therefore, there is an urgent need for those skilled in the art to develop a new IOL suspension structure.

### SUMMARY

An objective of the present disclosure is to provide an IOL suspension structure that solves the problem in existing devices where curved needles on a first movable stage often fail to realign with connecting tubes on a second movable stage after passing through the eyeball and an IOL, adversely affecting surgical efficiency.

To solve the above technical problems, the present disclosure employs the following technical solutions:

an IOL suspension structure includes a first fixing plate and a second fixing plate. The first fixing plate is fixedly connected to the second fixing plate through guide columns, a first moving plate and a second moving plate are arranged between the first fixing plate and the second fixing plate, and the first moving plate and the second moving plate are slidably matched with the guide columns; curved needles are arranged on a left end face of the first moving plate through clamping mechanisms, and the clamping mechanisms matched with the curved needles are arranged on a right end face of the second moving plate; and auxiliary holders matched with the curved needles are arranged between the first fixing plate and the second fixing plate.

In a further description, guide plates are fixedly arranged between the first fixing plate and the second fixing plate, sliding grooves are disposed on the guide plates, and bottom ends of the auxiliary holders are matched with the sliding grooves of the guide plates via sliding blocks.

In a further description, third lead screws are arranged inside the sliding grooves of the guide plates, the third lead screws are threadedly matched with the sliding blocks, and the third lead screws are rotatably matched with the first fixing plate and the second fixing plate.

In a further description, each auxiliary holder includes a cylindrical barrel, a rotating drum, and a movable slide bar; and the cylindrical barrel is fixedly arranged on the sliding block, the rotating drum is rotatably arranged inside the cylindrical barrel via a swivel, threads matched with the movable slide bar are arranged on an inner wall of the rotating drum, and a limit groove matched with the movable slide bar is disposed on the sliding block.

In a further description, a fixing wire matched with the curved needle is arranged at a bottom end of the movable slide bar, and the fixing wire is bent into a loop at the bottom end of the movable slide bar. In a further description, one end of the fixing wire is fixedly connected to the movable slide bar, and the other end of the fixing wire is routed through an internal channel of the movable slide bar and is connected to a pull ring located at an outer end of a lateral hole on the cylindrical barrel.

In a further description, a lock nut matched with the fixing wire is arranged at the lateral hole on the cylindrical barrel. In a further description, a first lead screw is rotatably connected to a right end face of the first moving plate, and the first lead screw is threadedly matched with the first fixing plate; and a second lead screw is rotatably connected to a left end face of the second moving plate, and the second lead screw is threadedly matched with the second fixing plate.

In a further description, the clamping mechanisms include movable clamping blocks and stationary clamping blocks; and the movable clamping blocks and the stationary clamping blocks are arranged inside mounting grooves of the first moving plate, and fixing screw rods matched with the movable clamping blocks are arranged on the first moving plate.

In a further description, two curved needles are arranged, each operatively associated with the corresponding auxiliary holder.

Compared with the related art, the present disclosure has the following beneficial effects.

The IOL suspension structure incorporates an auxiliary holder for each curved needle. The fixing wire on the auxiliary holder can guide the curved needle, facilitating its alignment with the clamping mechanism on the second moving plate after passing through the eyeball and the IOL, thereby improving surgical efficiency. The loop formed by the fixing wire on the auxiliary holder can be cinched tight by pulling the pull ring, allowing for direct extraction of the curved needle via the auxiliary holder in emergency scenarios, which enhances the operational reliability of the device. The movable slide bar on the auxiliary holder can be moved vertically through the rotating drum and adjusted horizontally through the third lead screw, ensuring operational flexibility of the device and facilitating manual intervention on the curved needle. In general, the IOL suspension structure is ingeniously designed, functionally practical, and structurally simple. It effectively solves the problem in existing devices where curved needles on a first movable stage often fail to realign with connecting tubes on a second movable stage after passing through the eyeball and the IOL, significantly compromising surgical efficiency.

### BRIEF DESCRIPTION OF THE DRAWINGS

The present disclosure is further described below with reference to the accompanying drawings.

FIG. 1 is a front view of an IOL suspension structure of the present disclosure;

FIG. 2 is a bottom view of the IOL suspension structure of the present disclosure;

FIG. 3 is a schematic structural diagram of an auxiliary holder and a guide plate;

FIG. 4 is a bottom view of the IOL suspension structure of the present disclosure (without the auxiliary holder and the guide plate);

FIG. 5 is a schematic structural diagram of a curved needle;

FIG. 6 is a schematic structural diagram of a clamping mechanism;

FIG. 7 is a front view of the auxiliary holder;

FIG. 8 is a cross-sectional view of FIG. 7; and

FIG. 9 is a schematic structural diagram of a movable slide bar.

Reference numerals and denotations thereof: 1-first fixing plate; 101-guide column; 2-second fixing plate; 3-first moving plate; 4-second moving plate; 5-first lead screw; 6-second lead screw; 7-auxiliary holder; 701-guide plate; 702-sliding groove; 703-third lead screw; 704-sliding block; 705-cylindrical barrel; 706-rotating drum; 707-swivel; 708-movable slide bar; 709-fixing wire; 710-pull ring; 711-lock nut; 8-curved needle; 9-clamping mechanism; 901-stationary clamping block; 902-movable clamping block; and 903-fixing screw rod.

### DETAILED DESCRIPTION

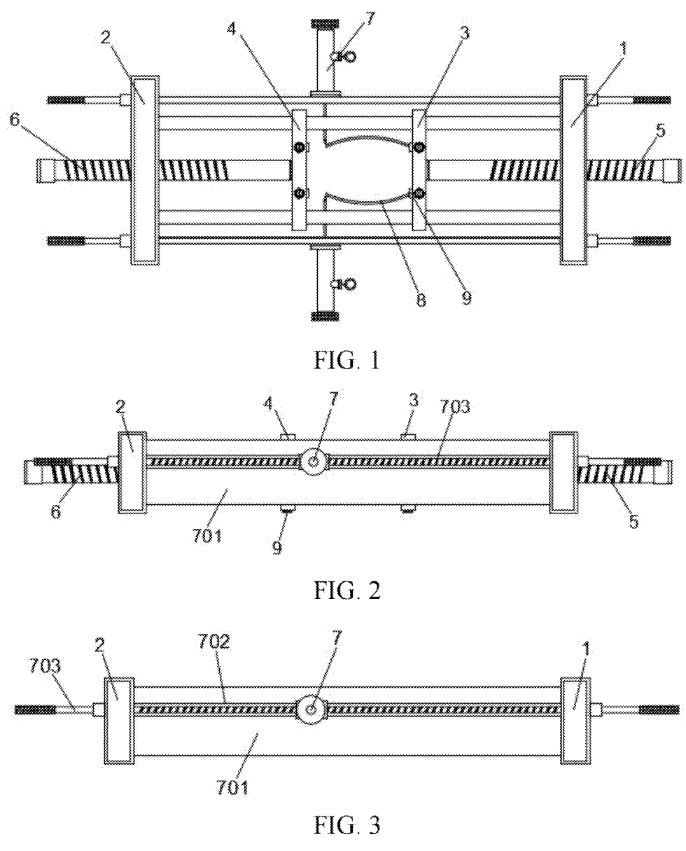

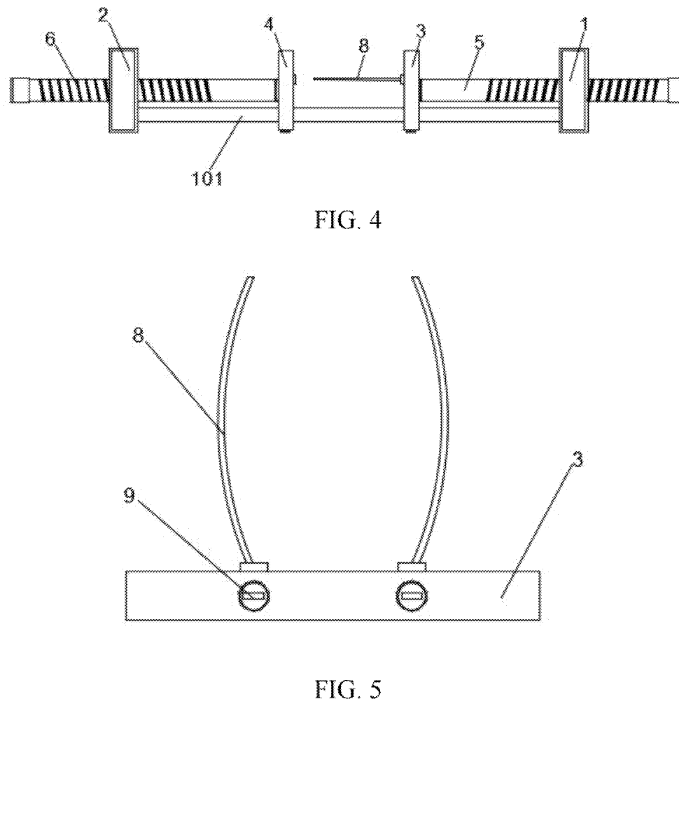

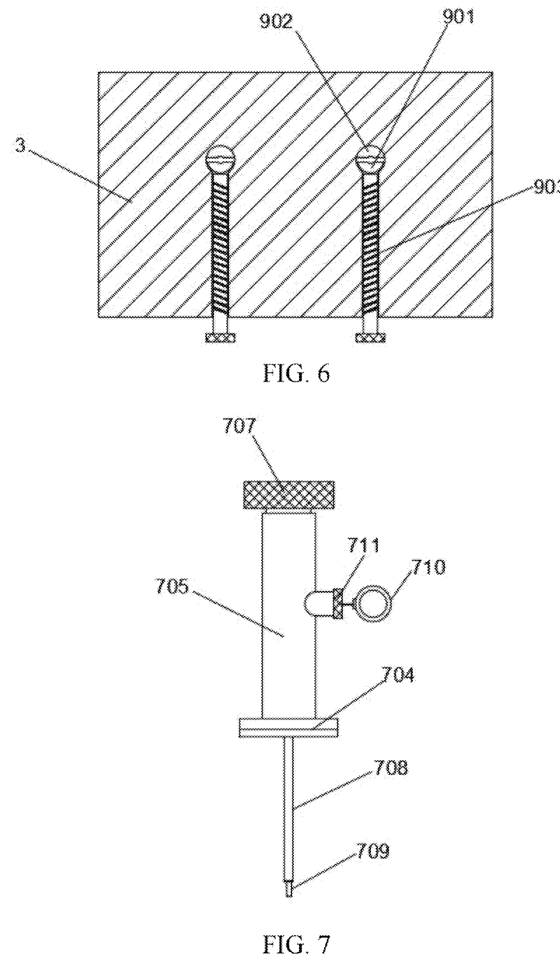

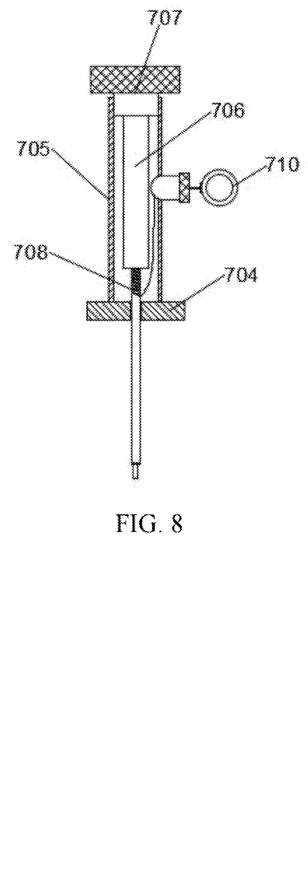

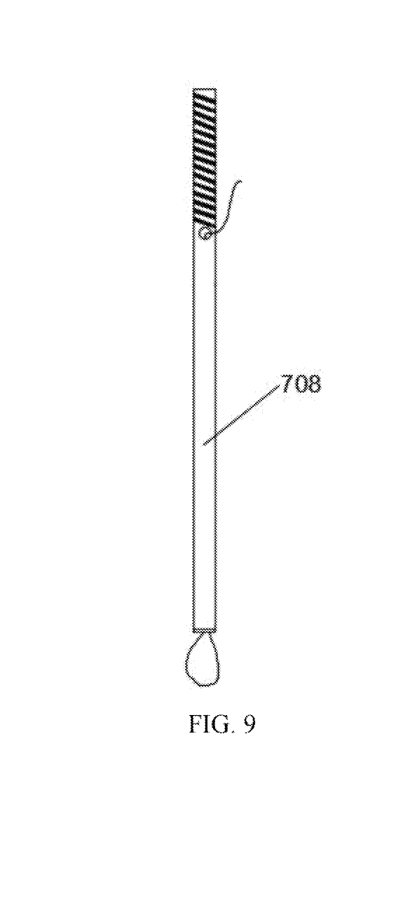

Referring to FIGS. 1-9, an IOL suspension structure includes a first fixing plate 1 and a second fixing plate 2, and the first fixing plate 1 is fixedly connected to the second fixing plate 2 through two guide columns 101.

A first moving plate 3 and a second moving plate 4 are arranged between the first fixing plate 1 and the second fixing plate 2, and the first moving plate 3 and the second moving plate 4 are slidably matched with the guide columns 101. The first moving plate 3 is arranged on one side close to the first fixing plate 1, and the second moving plate 4 is arranged on one side close to the second fixing plate 2. A first lead screw 5 is rotatably connected to a right end face of the first moving plate 3, and the first lead screw 5 is threadedly matched with the first fixing plate 1. A second lead screw 6 is rotatably connected to a left end face of the second moving plate 4, and the second lead screw 6 is threadedly matched with the second fixing plate 2.

Curved needles 8 are arranged on a left end face of the first moving plate 3 through clamping mechanisms 9, and the clamping mechanisms 9 matched with the curved needles 8 are arranged on a right end face of the second moving plate 4. The clamping mechanisms 9 include movable clamping blocks 902 and stationary clamping blocks 901; and the movable clamping blocks 902 and the stationary clamping blocks 901 are arranged inside mounting grooves of the first moving plate 3, and fixing screw rods 903 matched with the movable clamping blocks 902 are arranged on the first moving plate 3. The clamping mechanisms 9 can clamp and release the curved needles 8 by rotating the fixing screw rods 903.

Auxiliary holders 7 matched with the curved needles 8 are arranged between the first fixing plate 1 and the second fixing plate 2. Two curved needles 8 are arranged, each operatively associated with the corresponding auxiliary holder 7.

Specifically, guide plates 701 are fixedly arranged between the first fixing plate 1 and the second fixing plate 2, sliding grooves 702 are disposed on the guide plates 701, and bottom ends of the auxiliary holders 7 are matched with the sliding grooves 702 of the guide plates 701 via sliding blocks 704.

Third lead screws 703 are arranged inside the sliding grooves 702 of the guide plates 701, the third lead screws 703 are threadedly matched with the sliding blocks 704, and the third lead screws 703 are rotatably matched with the first fixing plate 1 and the second fixing plate 2.

Each auxiliary holder 7 includes a cylindrical barrel 705, a rotating drum 706, and a movable slide bar 708. The cylindrical barrel 705 is fixedly arranged on the sliding block 704, the rotating drum 706 is rotatably arranged inside the cylindrical barrel 705 via a swivel 707, threads matched with the movable slide bar 708 are arranged on an inner wall of the rotating drum 706, and a limit groove matched with the movable slide bar 708 is disposed on the sliding block 704.

A fixing wire 709 matched with the curved needle 8 is arranged at a bottom end of the movable slide bar 708, and the fixing wire 709 is bent into a loop at the bottom end of the movable slide bar 708.

One end of the fixing wire 709 is fixedly connected to the movable slide bar 708, and the other end of the fixing wire 709 is routed through an internal channel of the movable slide bar 708 and is connected to a pull ring 710 located at an outer end of a lateral hole on the cylindrical barrel 705. A lock nut 711 matched with the fixing wire 709 is arranged at the lateral hole on the cylindrical barrel 705. When in use, a size of the loop formed by the fixing wire 709 is adjusted by pulling the pull ring 710. By rotating the lock nut 711, the fixing wire 709 is secured in position within the lateral hole on the cylindrical barrel 705.

A working process of the present disclosure is as follows.

In step S1, proximal ends of the two sets of curved needles 8 are interconnected with a suture. The curved needles 8 are oriented with two ends facing upward, and suture-passing ends are fixed to the first moving plate 3 by the clamping mechanisms 9.

In step S2, the above structure is positioned at a location corresponding to the human eye, and the second fixing plate 2 is fixed to one side of the nasal bridge. Subsequently, the first lead screw 5 is rotated to drive the first moving plate 3 to move leftward. At this time, the curved needles 8 sequentially penetrate one side of the eyeball, a fixation point of the IOL, and the other side of the eyeball. The needles are secured by the clamping mechanisms 9 on the second moving plate 4, and the clamping mechanisms 9 on the first moving plate 3 are released.

In step S3, the second lead screw 6 is rotated to drive the second moving plate 4 to move leftward, thereby pulling the curved needles 8 and extracting the needles from the eyeball.

In step S4, the suture is tied with a knot, and the entire structure is removed.

During S2, if the curved needles 8 fail to align with the clamping mechanisms 9 on the second moving plate 4, the curved needles 8 may be maneuvered to first pass through the loops formed by the fixing wires 709 on the movable slide bars 708 of the auxiliary holders 7.

The movable slide bars 708 on the auxiliary holders 7 of the present disclosure's IOL suspension structure can be moved vertically through the rotating drums 706 and adjusted horizontally through the third lead screws 703, effectively ensuring operational flexibility of the device and facilitating manual intervention for orientation adjustment of the curved needles 8. The fixing wires 709 on the auxiliary holders 7 can guide the curved needles 8, facilitating the alignment with the clamping mechanisms 9 on the second moving plate 4 after passing through the eyeball and the IOL, thereby enhancing surgical efficiency.

The loops formed by the fixing wires 709 on the auxiliary holders 7 of the present disclosure's IOL suspension structure can be tightened or loosened via the pull rings 710. This configuration allows the movable slide bars 708 to lock onto and sleeve the curved needles 8 even in the absence of anteroposterior mobility. Moreover, in emergency scenarios, the curved needles 8 can be extracted directly through the auxiliary holders 7, thereby significantly enhancing the operational reliability of the device.

The foregoing embodiments are merely to describe preferred implementations of the present disclosure, rather than limiting the scope of the present disclosure. Without departing from the design spirit of the present disclosure, a person skilled in the art may make any modifications or improvements to the technical solutions of the present disclosure. Such modifications or improvements fall within the scope of protection defined by the claims of the present disclosure.

1. An intraocular lens (IOL) suspension structure, comprising a first fixing plate (1) and a second fixing plate (2), wherein the first fixing plate (1) is fixedly connected to the second fixing plate (2) through guide columns (101), a first moving plate (3) and a second moving plate (4) are arranged between the first fixing plate (1) and the second fixing plate (2), and the first moving plate (3) and the second moving plate (4) are slidably matched with the guide columns (101); curved needles (8) are arranged on a left end face of the first moving plate (3) through clamping mechanisms (9), and the clamping mechanisms (9) matched with the curved needles (8) are arranged on a right end face of the second moving plate (4); and auxiliary holders (7) matched with the curved needles (8) are arranged between the first fixing plate (1) and the second fixing plate (2).

2. The IOL suspension structure according to claim 1, wherein guide plates (701) are fixedly arranged between the first fixing plate (1) and the second fixing plate (2), sliding grooves (702) are disposed on the guide plates (701), and bottom ends of the auxiliary holders (7) are matched with the sliding grooves (702) of the guide plates (701) via sliding blocks (704).

3. The IOL suspension structure according to claim 2, wherein third lead screws (703) are arranged inside the sliding grooves (702) of the guide plates (701), the third lead screws (703) are threadedly matched with the sliding blocks (704), and the third lead screws (703) are rotatably matched with the first fixing plate (1) and the second fixing plate (2).

4. The IOL suspension structure according to claim 1, wherein each auxiliary holder (7) comprises a cylindrical barrel (705), a rotating drum (706), and a movable slide bar (708); and the cylindrical barrel (705) is fixedly arranged on the sliding block (704), the rotating drum (706) is rotatably arranged inside the cylindrical barrel (705) via a swivel (707), threads matched with the movable slide bar (708) are arranged on an inner wall of the rotating drum (706), and a limit groove matched with the movable slide bar (708) is disposed on the sliding block (704).

5. The IOL suspension structure according to claim 4, wherein a fixing wire (709) matched with the curved needle (8) is arranged at a bottom end of the movable slide bar (708), and the fixing wire (709) is bent into a loop at the bottom end of the movable slide bar (708).

6. The IOL suspension structure according to claim 5, wherein one end of the fixing wire (709) is fixedly connected to the movable slide bar (708), and the other end of the fixing wire (709) is routed through an internal channel of the movable slide bar (708) and is connected to a pull ring (710) located at an outer end of a lateral hole on the cylindrical barrel (705).

7. The IOL suspension structure according to claim 6, wherein a lock nut (711) matched with the fixing wire (709) is arranged at the lateral hole on the cylindrical barrel (705).

8. The IOL suspension structure according to claim 2, wherein a first lead screw (5) is rotatably connected to a right end face of the first moving plate (3), and the first lead screw (5) is threadedly matched with the first fixing plate (1); and a second lead screw (6) is rotatably connected to a left end face of the second moving plate (4), and the second lead screw (6) is threadedly matched with the second fixing plate (2).

9. The IOL suspension structure according to claim 1, wherein the clamping mechanisms (9) comprise movable clamping blocks (902) and stationary clamping blocks (901); and the movable clamping blocks (902) and the stationary clamping blocks (901) are arranged inside mounting grooves of the first moving plate (3), and fixing screw rods (903) matched with the movable clamping blocks (902) are arranged on the first moving plate (3).

10. The IOL suspension structure according to claim 1, wherein two curved needles (8) are arranged, each operatively associated with the corresponding auxiliary holder (7).
